(19) United States
(12) Patent Application Publication
(10) Pub. No.: US 2008/0097270 A1
(43) Pub. Date: Apr. 24, 2008

(54) Resilient hemostasis devices

(76) Inventors: David S. Utterberg, Seattle, WA (US); David Bell, Grayslake, IL (US); William J. Schnell, Libertyville, IL (US)

Correspondence Address: SEYFARTH SHAW LLP, 131 S. DEARBORN ST., SUITE 2400, CHICAGO, IL, 60603-5803, US

(21) Appl. No.: 11/510,383

(22) Filed: Aug. 25, 2006

(51) Int. Cl. A61F 13/15

(52) U.S. Cl. 602/42; 602/48

(57) ABSTRACT

A soft hemostasis pad of elastomeric material typically having a hardness of no more than about 50 Shore 00 and an upper wall having a raised profile, such as a convex shape. The pad has a central aperture extending therethrough, and a slit that extends through the pad thickness between the central aperture and the periphery of the pad.

### BACKGROUND OF THE INVENTION

In U.S. Patent Application Publication US/2006/0079823 A1, entitled Injection and Hemostasis Site, soft, hemostasis pads are disclosed, made of an elastomeric material having a hardness of no more than about durometer 50 Shore 00. This is a very soft elastomer, providing hemostasis before and after the withdrawal of needles and catheters from the skin, for example the large fistula needles used in hemodialysis, or catheters for femoral or other arterial sticks. Used as a substitute for gauze, the pads may create hemostasis of a fistula puncture or the like with much less loss of blood, and with much less pressure required to halt the blood flow, when compared with gauze. It is believed that this reduction in pressure which can be provided by such resilient hemostasis devices reduces damage to the fistula, a graft, arteries or the like, for longer life thereof and shorter hemostasis times.

Also, Master Seal™ hemostasis gel pads, sold by Medisystems Corporation, utilize raised-profile, non-porous, optically clear, flat-bottomed, soft elastomer pads for hemostasis before and after the withdrawal of medical, sharp needles from the vascular system.

By this invention, improvements are provided in the field of hemostasis for catheters and needle systems, both during implantation or cannulation and indwelling thereof, and also after withdrawal, reducing both blood seepage while a percutaneous catheter or other cannula, such as a sharp or dull needle, is emplaced in the patient, and optionally providing sealing hemostasis after withdrawal thereof.

### DESCRIPTION OF THE INVENTION

By this invention, a soft, hemostasis pad of elastomeric material having a periphery and, in some embodiments, a hardness of no more than about 50 Shore 00, is provided. The pad has an upper wall that comprises a raised profile that may have a convex shape in some embodiments, with a typically flat, non-porous bottom. The pad may have a central aperture extending therethrough, and a slit that laterally extends through the pad thickness between the central aperture and the periphery. The upper, raised profile wall may typically be generally convex, such as of dome shape, curving in two dimensions, or the convex wall may be of inverted U-shaped cross section, curving like an inverted trough in one dimension, so that the cross-sectional shape is substantially uniform along its length, for example. A “raised profile” has a central portion that is higher than at least some peripheral portions.

Typically, the pad may be made of a tacky, resilient material, so that the slit may be opened to laterally insert a typically percutaneous catheter or the like into the central aperture, while the catheter is typically implanted in the patient. The slit may then be reclosed, and held closed by tack adhesion because of the natural tack of the soft elastomeric material. Thus, the annular junction between the catheter and the skin of the patient, which is a site for infection and bleeding, may be sealed by the hemostasis pad of this invention.

Typically, the outer diameter of the catheter being enclosed by the pad aperture may be greater than the unstressed diameter of the pad central aperture by about 0.1 mm to 5 mm. Thus, there is stretching tension imposed by the catheter on the pad, to effect a seal around the catheter annular portion in the central aperture, while at least most of the slit may still be reclosed by the tack adhesion. In other words, the difference in the outer diameter of the catheter and the diameter of the central aperture is enough to provide a pressure between the catheter wall and the central aperture wall, but this pressure is preferably insufficient to substantially break the tack adhesion of the reclosed slit in the described pad as it surrounds the catheter. Adhesive tape may also be employed to maintain an annular seal of the pad around the catheter.

The hemostasis pads described herein may be made of a substantially nonabsorbent, typically transparent, pore-free material (at least at the bottom thereof), although nontransparent and/or porous (typically closed-cell) materials may be used, if desired.

In some embodiments, the hardness may be no more than about 30 or 35 Shore 00. For example, a currently favored material for manufacturing the pad is Gel Concepts thermoplastic rubber compound, which is a proprietary, oil-plasticized styrene block copolymer elastomer, manufactured by Gel Concepts L.L.C. of Whippany, N.J., particularly Product No. 4125. This is a transparent material, very soft, with a Shore 00 hardness of about 14. Another useful candidate material is Versaflex RTM CL 2003 X thermoplastic rubber compound, manufactured by GLS Corp. of McHenry, Ill., having a Shore 00 hardness of about 29, and having other physical parameters as described in the above-cited Patent Application Publication No. US2006/0079823 A1, the disclosures of which are incorporated by reference herein.

If desired, pads in accordance with this invention may carry a medicament, particularly to be present on and near the generally flat and non-porous bottom, and the central aperture wall, of the above-described pad. Such a medicament may be an antiseptic of any desired kind. Particularly, commercially available silver nanoparticles may be used, to be incorporated in effective concentration into the formulation of the material comprising the pad, but such nanoparticles do not strongly reduce the transparency of the pad, so that the pad may have antiseptic characteristics and remain transparent. Any other desired antiseptic may be included, for example, alcohols such as ethanol or isopropyl alcohol, and other known antiseptics, either applied to the bottom and other surfaces of the pad, or incorporated in the pad material itself for similar effect at the bottom and other surfaces.

Durometer measurements made herein are in accordance with ASTM Durometer Hardness Standard D-2240-03.

In some embodiments, the pad may have a generally circular periphery of a diameter of at least 4 times and preferably no more than 10 times the diameter of the retained catheter diameter at the annular seal point. The pad may also have a substantially cylindrical, central aperture, as described above, having a diameter of about 0.50 times to 0.95 times the diameter of the retained catheter diameter at the annular seal point. The maximum thickness of the convex or other raised profile pad may typically be at the center of the pad, with the pad maximum thickness being from at least about 1 and preferably no more than 5 times the diameter of the retained catheter diameter at the annular seal point. While the central aperture may be perpendicular to the generally flat bottom, it may also have an axis that defines an angle to the generally flat bottom ranging from typically about 20° to 90°. For example, the angle of the central aperture may be 30° to 80°, to accommodate catheters that are entering through the skin of the patient at such angles other than 90°, when that is desired. Such a central aperture may still have a cylindrical cross section, even though it extends at an acute angle of less than 90° to the substantially flat bottom of the pad.

It is also of advantage for the elastomeric material of the pad to contain a liquid plasticizer such as a mineral or other pharmaceutically acceptable oil, so that the material of the pad does not significantly adhere to a scab formed by bleeding under the solid bottom of the pad, as the pad rests on the skin of the patient. Thus, the pad is easily removed generally without tearing the scab, after the need to provide hemostasis is no longer present.

Although in some embodiments a generally flat bottom will best conform to the patient's skin at the cannulation or other percutaneous entry site, a slightly concave or convex bottom may better conform to the curving topography of a cannulation or percutaneous entry site on an arm, leg, armpit, or the like. Such a concave or convex shape is deemed “generally flat”.

Further by this invention, a method is provided which comprises: placing a soft, resilient, pore-free hemostasis pad having: an upper surface, typically having a raised profile, a periphery, a central aperture, and a slit extending between the periphery and the aperture, on the skin of a patient. A catheter or the like is extending through the patient's skin, and the pad is placed so that the catheter passes through the slit from the periphery and occupies the central aperture thereof. Then, the slit is substantially closed by tack adhesion, with or without adhesive tape assistance to suppress bleeding around the catheter.

The pad may comprise, as before, an elastomer having a hardness of no more than about 50 Shore 00, and preferably no more than about 30 Shore 00. As before, the pad's raised profile may be a dome or other convex shape, with a skin conforming bottom, so that when gentle, downward pressure is exerted on the top of the dome, it is efficiently transmitted to the bottom, providing a low pressure, typically less than is generally provided with cotton patch or swab hemostasis. Because of the softness of the pad, the elastomer material obstructs pathways on the skin surrounding the percutaneous catheter (or a sharp needle, or a skin injury from any source) preventing the flow of blood away from the injury site where typically a catheter or needle is present. Typically, the material of the pad is of approximately the softness of skin, which greatly improves the sealing ability, when compared with other, harder materials.

As disclosed, the central aperture may have an inner diameter that is from about 0.5 times to 0.95 times the outer diameter of the catheter or other cannula that it surrounds, the inner diameter being measured when the pad is unstressed condition. Further, as described above, the pad may be transparent. An antiseptic, as before, may be present on the pad bottom. Optionally the antiseptic is an integral part of the formulation of the entire pad. Silver nanoparticles, as stated before, may be used.

### DESCRIPTION OF THE DRAWINGS

In the drawings, FIG. 1 is a perspective view of the pad of this invention.

FIG. 2 is a plan view of the pad of FIG. 1.

FIG. 3 is an elevational view of the pad of FIGS. 1 and 2.

FIG. 4 is a perspective view, showing how the pad of FIGS. 1-3 may be applied to the skin and placed to surround a catheter that is implanted therein, extending through the skin.

FIG. 5 is a perspective view showing how the pad of this invention can be reclosed in surrounding relationship of the implanted catheter of FIG. 4.

FIG. 6 is a plan view of another design of the pad of this invention.

FIG. 7 is a side elevational view of the pad of FIG. 6, viewed along its longest dimension.

FIG. 8 is an end elevational view of the pad of FIG. 6.

### DESCRIPTION OF SPECIFIC EMBODIMENTS

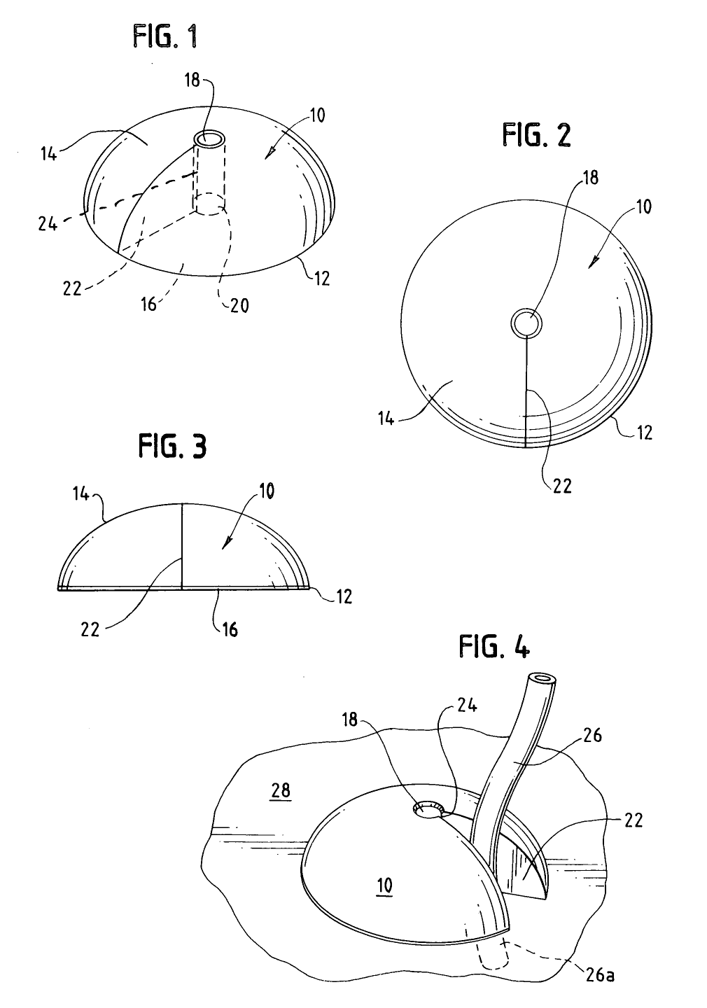

Referring to FIGS. 1-5, a soft, hemostasis pad of elastomeric material 10 is shown. Pad 10 has a periphery 12, and is made, typically by molding, of an elastomeric material having a hardness on the order of 30 Shore 00. Specifically, Gel Concepts oil-plasticized thermoplastic elastomer is a desirable candidate, as described above.

Pad 10 defines an raised profile upper wall 14 of convex, domed shape, and also defines a substantially flat, non-porous bottom 16. Pad 10 is solid and transparent, having a central aperture 18, open at both the top and the bottom 20. Additionally, slit 22 extends through the pad thickness between central aperture 18 and periphery 12 so that the entire length of central aperture 18 is opened at slit inner end 24 for access by a catheter 26.

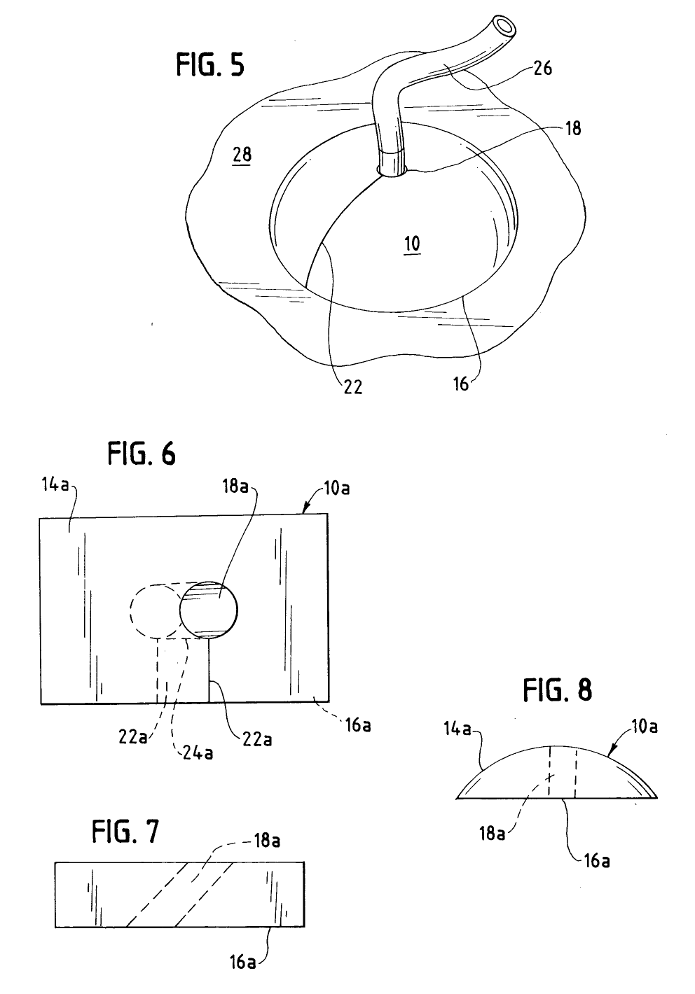

This is illustrated in FIG. 4, in which percutaneous catheter 26, implanted in a patient, and extending through the patient's skin 28 (so that a portion 26a of catheter 26 is under the skin 28 as is well known). The aperture in the skin where a catheter 26 joins the skin and extends therethrough is an area where blood can seep out onto the skin, and it becomes a site for infection, particularly with a long-term indwelling catheter. By this invention, pad 10 can be placed to surround an indwelling catheter 26 by opening of slit 22 as shown, which opens inner slit line 24 running the length of central aperture 18, to permit central aperture 18 to laterally receive catheter 26 and to surround it, as shown in FIG. 5. Slit 22 can be reclosed, being held together by the natural tack of the soft, resilient material of which pad 10 is made, as pad 10 rests with its bottom surface 16 resting on the skin 28. Thus, slit 22 is opened to laterally insert catheter 26 into central aperture 18. Then slit 22 is reclosed, and remains in reclosed position by natural tack adhesion. Also, pad 10 may be taped onto the skin to provide additional retention of the pad on the skin, and to assist in the maintenance of the reclosed condition of slit 22.

In this embodiment, the outer diameter of catheter 26 may be about 3 mm to 12 mm and greater than the unstressed diameter of the central aperture 18, which may be about 0.5 times to 0.95 times the catheter diameter. Thus, a compression seal is provided around catheter 26 within central aperture 18. The seepage of fluids from the catheter entrance site through the skin is sealed off and suppressed.

A medicament may be present on the flat, solid bottom surface 16, such as an antimicrobial agent, or another healing aid. If desired, this may be accomplished by placing the antimicrobial agent in the formulation of the entire pad material, so that it is present on all surfaces and in the interior of pad 10. For example, an effective amount (such as 0.001 to 0.1 weight percent) of colloidal silver may be added to the pad formulation, providing antimicrobial conditions on all surfaces of pad 10 including bottom surface 16, and the inner lumen surface of central aperture 18. The outer, circular periphery of bottom surface 16 may have a diameter of about 4 to 10 times the outer diameter of the catheter at the annular seal point, and the maximum thickness of pad 10 may be about 1 to 5 times the outer diameter of the catheter at the annular seal point, in this embodiment.

In the embodiment of FIGS. 1-5, central aperture 18 extends through pad 10 at a perpendicular angle to flat bottom 16. If desired, this angle may be varied, as described above, to accommodate different angles of indwelling catheters as they enter the skin.

As stated before, because of the presence of an oil plasticizer in the preferred formulation used to make pad 10, the oil plasticizer suppresses adherence of pad 10 to any scab that is formed by bleeding under bottom surface 16, as pad 10 rests on skin 28.

Referring to FIGS. 6-8, another embodiment of the pad of this invention is provided.

Pad 10a may be made from a similar oil-plasticized, soft, transparent, elastomer material as in the previous embodiment, comprising a flat, bottom wall 16a and a solid, transparent body, also comprising a convex, upper wall 14a. However, in this circumstance, contrary to the previous embodiment in which the convex upper wall is a dome shape, curving in two dimensions, the curvature of convex, upper wall 14a is a curvature in one dimension, so that upper wall 14a is of the shape of an inverted trough, having substantially similar cross sections along its length, contrary to the previous embodiment. As one advantage of this structure, the basic body of pad 10a can be extruded through a die aperture which is typically of D shape, for simplicity of manufacture.

Pad 10a also defines central aperture 18a, as in the previous embodiment, extending through the thickness of pad 10a and open at both ends. If desired, as indicated in FIGS. 6 and 7, central aperture 18a is not perpendicular to flat bottom 16a, but defines a desired angle thereto, to accommodate and receive catheters that are implanted through the skin of a patient at a similar angle. Alternatively, central aperture 18a may be perpendicular to surface 16a, if that is desired.

Slit 22a then is placed through the thickness of pad 10a in a direction parallel to the direction of central aperture 18a, so at the inner surface 24a of slit 22a extends along the length of central aperture 18a to open it, so that a catheter may be placed laterally through slit 22a to occupy central aperture 18a, and be sealed and supported by pad 10a resting on the skin and surrounding the catheter.

If desired, pad 10a (or pad 10) may be placed around the catheter (or needle) before the catheter is inserted into the body and the pad bottom is then mated to the skin, as may be preferable in certain surgical procedures. Also pad 10a (or pad 10) may be placed around the catheter and onto the skin after the catheter is inserted into the patient as may be preferable in long dwelling catheters such as dialysis catheter.

As before, it is preferred for the catheter used to have an outer diameter slightly larger than the diameter of central aperture 18a, so that a compression seal is provided around the catheter, but slit 22a is capable of being reclosed and sealed together by tack adhesion, although this seal can be reinforced by taping of pad 10a on the skin, as in the previous embodiment.

It is also possible to provide a medicament such as colloidal silver to the formulation of pad 10a, if desired.

Thus, a pad for catheters and the like provides support and sealing at the skin entry site. The pads provide hemostasis and sealing against body fluid seepage, while the catheter remains in implanted condition. Also, the pad may be modified to have an antimicrobial characteristic, or any other medicament may be applied to the pad to serve its desired purpose.

The above has been offered for illustrative purposes only, and is not intended to limit the scope of the invention of this application, which is as defined in the claims below.

That which is claimed is:

1. A soft hemostasis pad of elastomeric material having a periphery and a hardness of no more than about 50 Shore 00, said pad having an upper wall of raised profile with a generally flat, non-porous bottom, said pad having a central aperture extending therethrough and a slit that extends through the pad thickness between said central aperture and said periphery.

2. The pad of claim 1 in which the pad is made of a tacky material, whereby said slit may be opened to laterally insert a canteter into said central aperture and then to reclose the slit by tack adhesion.

3. The pad of claim 2 that has a catheter occupying said central aperture, the inner diameter of the pad being 0.5 to 0.95 times, the unstressed diameter of the catheter to effect a seal around the catheter in the central aperture, while at least most of the slit is reclosed by said tack adhesion.

4. The pad of claim 1 which at least the bottom of which is made of a solid, transparent, pore-free material.

5. The pad of claim 2 in which the hardness is no more than about 35 Shore 00.

6. The pad of claim 1 in which a medicament is present on the flat, solid bottom.

7. The pad of claim 6 in which said medicament comprises silver nanoparticles distributed throughout the pad, said pad being transparent.

8. The paid of claim 1 in which the upper wall has a generally convex, raised profile.

9. The pad of claim 1 in which the upper wall has a generally convex, raised profile, said pad being transparent, and at least the bottom of said pad being pore-free.

10. The pad of claim 1 in which the central aperture has an axis that defines an angle to the substantially flat bottom of 20° to 900.

11. The pad of claim 1 in which the upper wall raised profile is a dome shape.

12. The pad of claim 1 in which said elastomeric material contains a liquid plasticizer, whereby the material does not significantly adhere to a scab formed by bleeding under the solid bottom as the pad rests on the skin of a patient.

13. The method which comprises:
placing a soft, resilient, pore-free hemostasis pad having a periphery, a central aperture, and a slit extending between the periphery and the aperture on the skin of a patient with a catheter extending through the patient's skin and occupying the central aperture, and substantially closing the slit by tack adhesion, to suppress bleeding around the catheter.

14. The method of claim 13 in which said pad comprises an elastomer having a hardness of no more than about 50 Shore 00, said pad having an upper wall of raised profile with a flat bottom.

15. The method of claim 14 in which the hardness is no more than about 35 Shore 00.

16. The method of claim 14 in which said upper wall is of convex shape, and said central aperture is positioned at substantially the thickest part of said pad.

17. The method of claim 13 in which said central aperture has an inner diameter which, in unstressed condition, is from 50 to 95 percent of the catheter outer diameter.

18. The method of claim 13 in which said pad is substantially transparent.

19. The method of claim 14 in which an antiseptic is present on the flat bottom.

20. A soft, solid, transparent, pore-free pad of elastomeric material having a periphery and a hardness of no more than about 50 Shore 00, said pad having an upper wall of convex shape with a substantially flat, solid bottom, said pad having a central aperture extending therethrough, and a slit that extends through the pad thickness between said central aperture and the periphery to permit the lateral installation of a catheter or the like in said central aperture, said pad comprising an elastomeric material that contains a liquid plasticizer, whereby the material does not significantly adhere to a scab formed by bleeding under the solid bottom as the pad rests on the skin of a patient.

21. The pad of claim 20 in which the hardness is no more than about 35 Shore 00.

22. The pad of claim 21 in which said pad contains an antimicrobial concentration of silver nanoparticles distributed at least within the bottom of the pad, said pad being substantially transparent.

23. The pad of claim 20 in which the upper wall convex shape is a dome shape.

24. The pad of claim 20 in which a catheter occupies said central aperture, the outer diameter of the catheter being slightly greater than the unstressed diameter of the central aperture, to effect a seal around the catheter in the central aperture while permitting at least most of the slit to be reclosed by said tack adhesion.
